(19) United States
(12) Patent Application Publication
(10) Pub. No.: US 2025/0208049 A1
(43) Pub. Date: Jun. 26, 2025

(54) OPTICAL DEVICE AND BIOSENSOR

(71) Applicant: KYOCERA CORPORATION, Kyoto (JP)

(72) Inventor: Tetsuya MATSUSHITA, Kyoto (JP)

(21) Appl. No.: 18/848,909

(22) PCT Filed: Mar. 1, 2023
(86) PCT No.: PCT/JP2023/007569

§ 371 Date: Sep. 20, 2024

(30) Foreign Application Priority Data
Mar. 30, 2022 (JP) 2022-055905

(51) Int. Cl. G01N 21/77; G01N 21/552

(52) U.S. Cl. CPC G01N 21/7703; G01N 21/552; G01N 2021/7723

(57) ABSTRACT

An optical device includes: a substrate to transmit light; a reflective layer on the substrate; an optical waveguide layer to propagate the light transmitted through the reflective layer or near-field light bled from the reflective layer, the optical waveguide layer being on the reflective layer and having a surface with a functional group immobilizing a capturing body that captures a specimen; and a wavelength adjustment layer on the substrate side, the optical waveguide layer side, or both the substrate side and the optical waveguide layer side of the reflective layer, and configured to shift a peak wavelength in a waveform indicating a first relationship between (i) a wavelength of the light and (ii) an intensity of the light reflected by a surface of the reflective layer on the optical waveguide layer side or a surface of the optical waveguide layer opposite to the reflective layer under a total reflection condition.

### CROSS-REFERENCE TO RELATED APPLICATIONS

This application is national stage application of International Application No. PCT/JP2023/007569, filed on Mar. 1, 2023, which claims priority to Japanese Application No. 2022-055905, filed Mar. 30, 2022.

### TECHNICAL FIELD

The present disclosure relates to an optical device that immobilizes a capturing body that captures a specimen, and a biosensor including the optical device.

### BACKGROUND OF INVENTION

As a biosensor for detecting a protein such as a virus protein (antigen), a biosensor using near-field light (evanescent light) has been known. The biosensor includes an optical device including a glass substrate, a reflective layer coated on the glass substrate, and an optical waveguide layer (dielectric layer) formed on the reflective layer. The biosensor includes a light incidence mechanism making light incident on the reflective layer from the substrate side of the optical device, and a light detection mechanism that detects reflected light that is the light reflected from the reflective layer. At this time, the light totally reflected by the upper surface of the reflective layer (the interface between the reflective layer and the optical waveguide layer) causes near-field light to bleed toward the optical waveguide layer. In the biosensor, the incident light partially or entirely propagates through the optical waveguide layer. Incident light is made incident on the optical waveguide layer at an incident angle at which the reflected light intensity (reflection intensity) decreases. When a specimen (detection target substance) is adsorbed or attached to the surface of the optical waveguide layer, the refractive index in the vicinity of the surface of the optical waveguide layer changes, and a change in the reflected light intensity caused by the change in the near-field light is read. Thus, the specimen is detected.

### CITATION LIST

### Patent Literature

Patent Document 1: WO 2007/029414

### SUMMARY

An optical device according to a non-limiting aspect of the present disclosure includes: a substrate configured to transmit light; a reflective layer disposed on the substrate; an optical waveguide layer configured to propagate the transmitted light transmitted through the reflective layer or near-field light bled from the reflective layer, the optical waveguide layer being located on the reflective layer and having a surface provided with a functional group configured to immobilize a capturing body configured to capture a specimen; and a wavelength adjustment layer located the substrate side, the optical waveguide layer side, or both the substrate side and the optical waveguide layer side of the reflective layer, and configured to shift a peak wavelength in a waveform indicating a first relationship between (i) a wavelength of the light and (ii) an intensity of the light reflected by a surface of the reflective layer on the optical waveguide layer side or a surface of the optical waveguide layer on a side opposite to the reflective layer under a total reflection condition.

### BRIEF DESCRIPTION OF THE DRAWINGS

FIG. 1 is a cross-sectional view illustrating an optical device according to a first embodiment of the present disclosure.

FIG. 2 is a cross-sectional view illustrating a state in which an antibody is immobilized on a silanol group exposed on a surface of an optical waveguide layer.

FIG. 3 is a schematic cross-sectional view illustrating a configuration example of a biosensor including the optical device.

FIG. 4 is a cross-sectional view illustrating a configuration of a prototype optical device.

FIG. 5 is an example of a graph showing the relationship between a reflection intensity of a specimen and a light source wavelength in the biosensor.

FIG. 6 is an example of a characteristic graph showing a difference in reflection intensity between two graphs in FIG. 5 with respect to the light source wavelength.

FIG. 7 is an example of a plurality of characteristic graphs corresponding to a film thickness of a wavelength adjustment layer.

FIG. 8 is an example of a plurality of characteristic graphs with the film thickness of the optical waveguide layer varied.

FIG. 9 is a cross-sectional view illustrating an optical device according to a second embodiment of the present disclosure.

FIG. 10 is a cross-sectional view illustrating an optical device according to a third embodiment of the present disclosure.

### DESCRIPTION OF EMBODIMENTS

In a known biosensor as described in Patent Document 1, the detection capability is determined by the selection of the film thicknesses and refractive indices of the reflective layer and the optical waveguide layer of the optical device, the incident angle, and the wavelength of the light source (hereinafter, referred to as the light source wavelength). Now, how the light source wavelength is selected will be described. A case will be considered in which in the biosensor, the reflection intensity is measured with a liquid containing a specimen and a liquid not containing a specimen being the measurement target, and with the light source wavelength selectively varied. In this case, a change (first change amount) in the reflection intensity having a first resonance point is obtained according to the optical characteristic (refractive index) of the liquid containing the specimen, and a change (second change amount) in the reflection intensity having a second resonance point is obtained according to the optical characteristic of the liquid not containing a specimen. The difference between the first change amount and the second change amount is the detection capability of the biosensor. In order to enhance the detection capability, the light source wavelength may be set to make the difference large. When the light source wavelength is set as described above, film design is required for the optical device to achieve high detection capability at the set light source wavelength. However, even when the reflective layer and the optical waveguide layer are manufactured with the film thickness and the refractive index selected, the wavelength suitable for detection at which the difference is maximized may be deviated from the set light source wavelength due to, for example, manufacturing variations. In order to investigate and correct the cause of such a wavelength deviation, it is necessary to check and correct the film thicknesses and refractive indices of the optical waveguide layer and the reflective layer, but this takes a lot of time.

The present disclosure provides an optical device whose optical characteristics can be readily adjusted to be suitable for the detection of a specimen having a particular optical characteristic.

With an optical device according to a non-limiting aspect of the present disclosure, the optical characteristic can be readily adjusted so as to be suitable for detection of a specimen having a particular optical characteristic.

An optical device and a biosensor according to an embodiment as an example of the present disclosure will be described below in detail with reference to the drawings. However, each of the figures, which will be referred to below, is a simplified representation of only main members necessary for description of the embodiments. Accordingly, the optical device and the biosensor may be provided with an optional component that is not illustrated in each of the referred drawings. In addition, the dimensions of members in the respective figures do not accurately represent the actual dimensions of constituent members, the dimensional ratio of respective members, or the like.

### First Embodiment

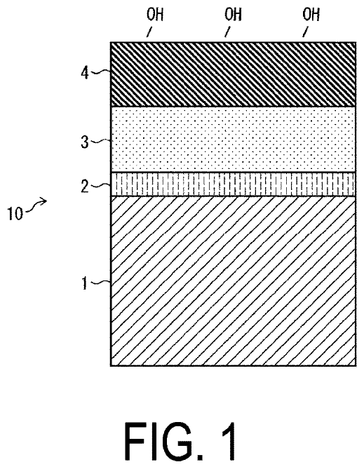

FIG. 1 is a cross-sectional view illustrating an optical device 10 according to a first embodiment of the present disclosure. The optical device 10 includes a substrate 1, a wavelength adjustment layer 2, a reflective layer 3, and an optical waveguide layer 4.

Substrate The substrate 1 transmits light used for detection. The material of the substrate 1 may be a translucent dielectric material such as a glass, a resin, a ceramic, or an insulator, or a translucent conductive material such as indium tin oxide (ITO). The substrate 1 may have a refractive index not less than 1.4 and not more than 1.65 when a wavelength of a light source 22 described below (hereinafter, referred to as light source wavelength) used at the time of detection of a specimen is, for example, 632.8 nm.

### Wavelength Adjustment Layer

The wavelength adjustment layer 2 is located on the substrate 1. Examples of the material of the wavelength adjustment layer 2 include SiON, SiN, and the like. When the wavelength of the light incident on the substrate 1 is varied, a peak wavelength (resonance point) at which the minimum reflection intensity in the reflective layer 3 is achieved is identified, based on the optical characteristics (mainly, refractive index) of the surface of the optical waveguide layer 4. The wavelength adjustment layer 2 shifts the wavelength of the resonance point. Specifically, the wavelength adjustment layer 2 shifts the peak wavelength in a waveform illustrating a first relationship between (i) the wavelength of light (the light source 22 described below) incident on the substrate 1 and (ii) the intensity of the light reflected by the surface of the reflective layer 3 on the optical waveguide layer 4 side or the surface of the optical waveguide layer 4 on the side opposite to the reflective layer 3 under a total reflection condition. The method of setting the film thickness of the wavelength adjustment layer 2 will be described below. The wavelength adjustment layer 2 may have a refractive index not less than 3 and not more than 3.7 when the light source wavelength is 632.8 nm, for example.

### Reflective Layer

The reflective layer 3 is located on the wavelength adjustment layer 2. As the material of the reflective layer 3, a chemically and physically stable metal thin film or semiconductor thin film can be used. The metal material used for the reflective layer 3 includes a metal selected from Groups 4 to 14 of the periodic table of elements, or an alloy mainly using such a metal. The semiconductor material may be a compound semiconductor composed of two or more types of elements, instead of a semiconductor composed of one type of element such as Si and Ge. The semiconductor may be any of p-type, n-type, and intrinsic semiconductors. The semiconductor material may be Si, amorphous Si (a-Si), crystalline Si (monocrystalline Si, polycrystalline Si, microcrystalline Si), or the like. For coating the substrate 1 with a thin film of a semiconductor material, vapor deposition, sputtering, various types of chemical vapor deposition (CVD), molecular beam epitaxy (MBE) and the like can be used. The reflective layer 3 may have a refractive index not less than 3.8 and not more than 4.5 when the light source wavelength is, for example, 632.8 nm.

### Optical Waveguide Layer

The optical waveguide layer 4 is located on the reflective layer 3, and has on the surface, a functional group (hereinafter, referred to as a specific functional group) for immobilizing a capturing body for capturing a specimen. The transmitted light transmitted through the reflective layer 3 or near-field light bled from the reflective layer 3 propagates through the optical waveguide layer 4. This near-field light is also called evanescent light, and is light bleeding into the optical waveguide layer 4 under the total reflection condition when light incident from the substrate 1 is reflected at the upper surface of the reflective layer 3 (the interface between the reflective layer 3 and the optical waveguide layer 4), where the incident angle of light to the reflective layer 3 is larger than the critical angle. The capturing body immobilized on the surface (upper surface) of the optical waveguide layer 4 on the side opposite to the reflective layer 3 changes its optical characteristics upon capturing the specimen. The resonance condition is changed and the intensity of the near-field light is changed in response to the change in the optical characteristics (mainly refractive index) on the surface of the optical waveguide layer 4. Thus, the presence of the specimen can be detected by optically detecting the change. When the near-field light bled to the optical waveguide layer 4 is used for detection of a specimen, the film thickness of the optical waveguide layer 4 is set to be approximately the same as the bleeding range of the near-field light to make the near-field light propagate in the vicinity of the surface of the optical waveguide layer 4. When light transmitted through the optical waveguide layer 4 is totally reflected by the upper surface of the optical waveguide layer 4 (the surface of the optical waveguide layer 4 opposite to the reflective layer 3) to cause bleed of the near-field light to the surface of the optical waveguide layer 4, the film thickness of the optical waveguide layer 4 may be set in a range in which the transmitted light can be totally reflected by the surface of the optical waveguide layer 4. The optical waveguide layer 4 is formed to be mainly composed of an oxide of a semiconductor material, a nitride of a semiconductor material, a carbide of a semiconductor material, or the like. Specifically, the material of the optical waveguide layer 4 includes SiO₂, SiNx, SiON, SiC, and the like. When these materials are used, a silanol group functioning as the specific functional group is exposed on the surface of the optical waveguide layer 4. The surface of the optical waveguide layer 4 may be chemically modified with a specific functional group such as —NH₂, —COOH, —SCN, a carboxyl group, a succinimide group, or a biotinyl group by using a silane coupling agent or the like. FIG. 1 illustrates a case where the silanol group is exposed on the surface of the optical waveguide layer 4. The optical waveguide layer 4 may have a refractive index not less than 1.4 and not more than 2.01 when the light source wavelength is, for example, 632.8 nm.

### Refractive Index and Film Thickness

The refractive indices of the wavelength adjustment layer 2, the reflective layer 3, and the optical waveguide layer 4 may satisfy the following relational expressions:

(refractive index of reflective layer 3)>(refractive index of wavelength adjustment layer 2)>(refractive index of optical waveguide layer 4); and

(refractive index of reflective layer 3)>(refractive index of wavelength adjustment layer 2)>(refractive index of substrate 1).

The film thickness of the wavelength adjustment layer 2 may be smaller than the film thickness of the reflective layer 3.

### Immobilization

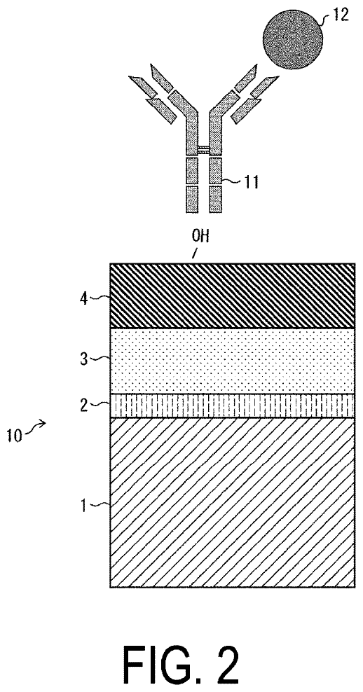

FIG. 2 is a cross-sectional view illustrating a state in which an antibody 11 is immobilized on the silanol group (specific functional group) exposed on the surface of the optical waveguide layer 4. Actually, when the antibody 11 is chemically bonded to a silanol group, the silanol group changes its structure as a result of the chemical bonding, but for convenience of explanation, the silanol group is illustrated as is in FIG. 2. This also applies to the other figures. Hereinafter, as illustrated in FIG. 2, a case where, for example, the antibody 11 as the capturing body is bonded to the silanol group will be described. An antigen 12 as a specimen interacts with the antibody 11.

### Biosensor

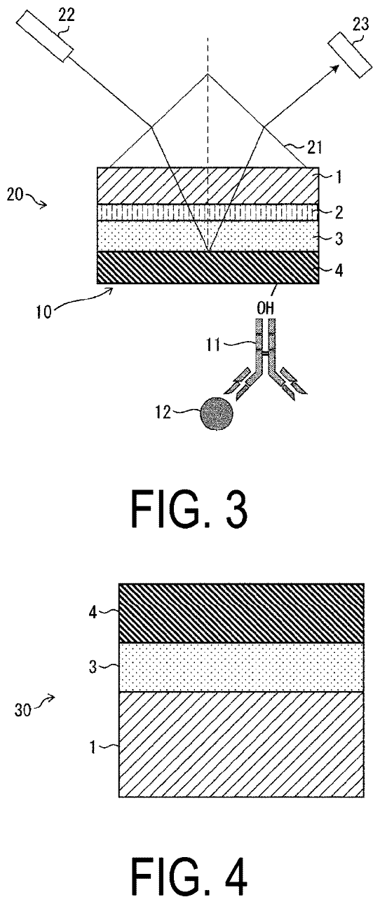

FIG. 3 is a schematic cross-sectional view illustrating a configuration example of a biosensor 20 including the optical device 10. The biosensor 20 illustrated in FIG. 3 includes the optical device 10, the light source 22, a prism 21, and a detector 23. The light source 22 and the prism 21 function as a light incidence mechanism that causes light to be incident on the reflective layer 3 from the surface of the substrate 1 of the optical device 10 opposite to the surface on which the reflective layer 3 is located. The detector 23 and the prism 21 function as a light detection mechanism that detects reflected light as a result of reflection of light made incident by the light incidence mechanism on the lower surface of the reflective layer 3 (the interface between the reflective layer 3 and the optical waveguide layer 4) or the lower surface of the optical waveguide layer 4 (the surface of the optical waveguide layer 4 on the side opposite to the reflective layer 3) under the total reflection condition. The reflected light reflected under the total reflection condition causes the near-field light to bleed out beyond the reflection surface. The biosensor 20 detects a change in the reflected light caused by the change in the near-field light to detect the specimen.

The prism 21 is brought into close contact with the substrate 1 via refractive index adjusting oil. For example, the light source 22 emits a laser beam toward the prism 21. The light used for the detection, which is not particularly limited as long as it is an electromagnetic wave, may be light in the infrared to ultraviolet range for the sake of easy handling. The detection capability of the biosensor 20 is determined by selecting the film thicknesses and refractive indices of the reflective layer 3 and the optical waveguide layer 4, as well as the incident angle and the light source wavelength. The laser beam incident at a specific incident angle activates an optical waveguide mode for propagation in the optical waveguide layer 4. The optical waveguide mode activation refers to a phenomenon in which incident light is absorbed without being totally reflected by the reflective layer 3 or both the reflective layer 3 and the optical waveguide layer 4, resulting in the intensity of reflected light being lower than the intensity of incident light. When the refractive index of the surface of the optical waveguide layer 4 changes under the condition of the optical waveguide mode activation, the change in the refractive index appears as a change in reflection intensity. The biosensor 20 detects the adsorption or lack of adsorption of the antigen 12 as a specimen to the antibody 11 immobilized on the surface of the optical waveguide layer 4. For this detection, a phenomenon is used in which the intensity of near-field light bleeding from the biosensor 20 changes due to a change in the refractive index in the vicinity of the surface including the antibody 11 caused by the adsorption of the antigen 12. The adsorption or lack of adsorption of the antigen 12 to the antibody 11 can be detected by monitoring a change in the intensity of the reflected light, affected by a change in the intensity of the near-field light, by the detector 23. When the antigen 12 is captured by the antibody 11 on the surface of the optical waveguide layer 4, a change in the refractive index occurs in or in the vicinity of the optical waveguide layer 4. The incident light source wavelength is set within a range of wavelengths causing a change in the refractive index. With such a setting, the reflection intensity sharply changes when the antigen 12 is captured.

Setting of Film Thickness of Wavelength Adjustment Layer Hereinafter, a method of setting the film thickness of the wavelength adjustment layer 2 of the optical device 10 will be described. Here, as an example, a case where the light source wavelength is 630 nm will be described. The method of setting the film thickness of the wavelength adjustment layer 2 includes: (1) a first step of measuring, for a prototype optical device 30 not including the wavelength adjustment layer 2, a first reflection intensity, obtained on the surface of the reflective layer 3 on the optical waveguide layer 4 side or the surface of the optical waveguide layer 4 opposite to the reflective layer 3, when light is incident on the substrate 1 with the antigen 12 captured by the antibody 11, and a second reflection intensity obtained on the surface of the reflective layer 3 on the optical waveguide layer 4 side or the surface of the optical waveguide layer 4 opposite to the reflective layer 3, when light is incident on the substrate 1 without the antigen 12 being captured, while varying the wavelength of the light; (2) a second step of identifying the peak wavelength in a characteristic waveform (characteristic graph) indicating a difference between the first reflection intensity and the second reflection intensity measured in the first step; and (3) a third step of setting the film thickness of the wavelength adjustment layer 2 with reference to a second relationship based on the peak wavelength identified in the second step. The second relationship is a relationship obtained in advance, and is a relationship between the peak wavelength in the characteristic waveform and each film thickness, for each of the optical devices 10 including the wavelength adjustment layers 2 with different film thicknesses.

First of all, for the prototype optical device 30 not including the wavelength adjustment layer 2, the first reflection intensity and the second reflection intensity are measured while varying the light source wavelength in each of the case where the antigen 12 is not captured by the antibody 11 and the case where the antigen 12 is captured by the antibody 11 (first step). The relationship between the first reflection intensity and the light source wavelength obtained in the first step corresponds to the first relationship described above.

The prototype optical device 30 will be described below. FIG. 4 is a cross-sectional view illustrating a configuration of the prototype optical device 30. Unlike the configuration of the optical device 10 of FIG. 1, the prototype optical device 30 does not include the wavelength adjustment layer 2, and the reflective layer 3 and the optical waveguide layer 4 are stacked in this order on the substrate 1.

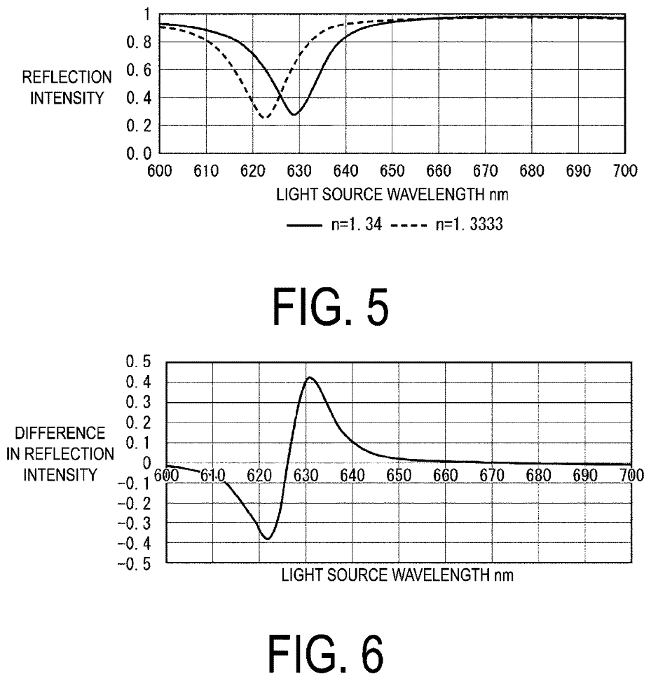

The results of measurement using the prototype optical device 30 instead of the optical device 10 of the biosensor 20 illustrated in FIG. 3 will be described. FIG. 5 is an example of a graph showing the relationship between the reflection intensity of the specimen and the light source wavelength in the biosensor. In FIG. 5, the horizontal axis represents the light source wavelength (nm), and the vertical axis represents reflection intensity (relative value). In FIG. 5, a broken line graph is a graph showing the second reflection intensity when the refractive index of the specimen is 1.3333, that is, when the comparison object is water in which the antigen 12 is not captured by the antibody 11. In FIG. 5, a solid line graph is a graph of the first reflection intensity (a graph of the first relationship) when water including the antigen 12 is used as the specimen and the antigen 12 is captured by the antibody 11 (when the refractive index is 1.34). As illustrated in FIG. 5, there is a resonance point at which the reflection intensity decreases to a minimum value depending on the difference in optical characteristics (mainly refractive index) on the surface of the optical waveguide layer 4.

Next, a characteristic waveform indicating a difference between the first reflection intensity and the second reflection intensity measured in the first step is obtained, and a peak wavelength in the characteristic waveform is identified (second step). FIG. 6 is an example of a graph (hereinafter, referred to as a characteristic graph) in which a difference in reflection intensity between the two graphs in FIG. 5 (a difference between the first reflection intensity and the second reflection intensity) is presented with respect to the light source wavelength. The vertical axis of FIG. 6 represents the difference in reflection intensity. From the characteristic graph in FIG. 6, it can be seen that when the capturing of the antigen 12 is detected, if the wavelength of the light sources 22 used for measurement (hereinafter, referred to as measurement wavelength) is set to be around 630 nm (which may be 632.8 nm), a maximum difference in reflection intensity exceeding 0.4 is obtained, whereby the presence of the antigen 12 can be detected with high sensitivity. Here, for example, when the measurement wavelength is set to 640 nm, the difference in the reflection intensity is about 0.1, which is small, meaning that the antigen 12 cannot be detected with high sensitivity.

When the measurement wavelength of the light source 22 of the biosensor is set and fixed to be around the peak wavelength based on the result on the characteristic graph, the film design of the prototype optical device 30 is preferably such that the detection capability at the measurement wavelength is high, that is, the peak wavelength of the characteristic graph of FIG. 6 coincides with the measurement wavelength. However, even when the optical device is manufactured by setting parameters such as the film thicknesses and the refractive indices of the reflective layer 3 and the optical waveguide layer 4 as well as the incident angle based on the result obtained with the prototype optical device 30, a peak wavelength suitable for measurement for the prototype optical device 30 (hereinafter, referred to as a preferable peak wavelength) may deviate from the set measurement wavelength due to manufacturing variations. For example, when the measurement wavelength is set to 630 nm, the preferable peak wavelength may be shifted to 635 nm. In this case, in order to investigate the cause of the deviation and correct the film design conditions of the optical device so that the preferable peak wavelength coincides with the measurement wavelength, it is necessary to check and set the refractive index and film thickness conditions of the reflective layer 3 and the optical waveguide layer 4, which takes a lot of time. When one parameter is changed, the preferable peak wavelength may be shifted more than expected due to the impact of such a change.

In view of the above, the optical device 10 according to the first embodiment includes the wavelength adjustment layer 2 as described above. By changing the film thickness of the wavelength adjustment layer 2, the preferable peak wavelength in the optical device 10 can be shifted to the measurement wavelength without changing the wavelength of the light source 22 and without changing the height of the difference (intensity) in reflection intensity at the preferable peak wavelength in the characteristic graph.

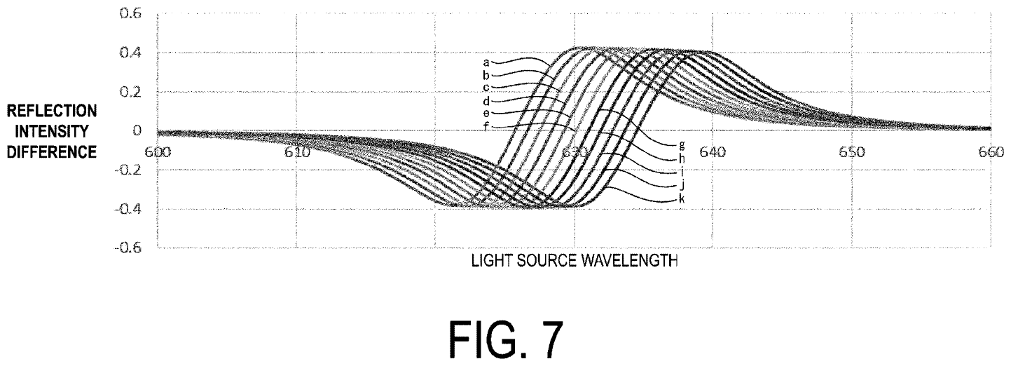

For setting the film thickness of the wavelength adjustment layer 2, the second relationship between the peak wavelength and the film thickness in each characteristic graph (the characteristic graph of the difference in reflection intensity with respect to the light source wavelength between cases where the specimen is present and absent) is obtained in advance for each of the cases of different film thicknesses of the wavelength adjustment layer 2 in the optical device 10. That is, the film thickness of the wavelength adjustment layer 2 can be identified by referring to the second relationship in the optical device 10 based on the preferable peak wavelength obtained by obtaining the characteristic graph for the prototype optical device 30. FIG. 7 illustrates examples of a plurality of characteristic graphs with the film thickness of the wavelength adjustment layer 2 varied. The wavelength adjustment layer 2 in this example is made of SiON and has a refractive index of 3.5. A biosensor is configured using the prototype optical device 30, and the incident angle of light is set to, for example, 67.5°. In FIG. 7, a, b, c, d, e, and f respectively indicate cases where the film thicknesses of the wavelength adjustment layer 2 are 0 nm, 1 nm, 2 nm, 3 nm, 4 nm, and 5 nm. Furthermore, g, h, i, j, and k respectively indicate cases where the film thicknesses are 6 nm, 7 nm, 8 nm, 9 nm, and 10 nm. It can be seen in FIG. 7 that an increase in the film thickness of the wavelength adjustment layer 2 results in shifting of the peak wavelength of the characteristic graph corresponding to each film thickness from that in the graph a toward the long wavelength side, but does not involve a change in the height of the peak (reflection intensity difference). Thus, it can be understood that by changing the film thickness of the wavelength adjustment layer 2, the peak wavelength can be adjusted while maintaining the height of the peak (reflection intensity difference).

In the optical device 10, by setting the film thickness of the wavelength adjustment layer 2 to make the peak wavelength shift toward the measurement wavelength, the peak wavelength can be adjusted without changing the reflection intensity difference, whereby the presence of the antigen 12 can be detected with high sensitivity. Therefore, correction to desired characteristics, for variations in the characteristics of the optical device 10 due to manufacturing variations, can be easily achieved through correction of the wavelength adjustment layer 2. For example, when the preferable peak wavelength of the prototype optical device 30 is 630 nm and the measurement wavelength of the light source 22 suitable for detecting the antigen 12 is 635 nm, the film thickness in the graph f with the peak wavelength of 635 nm is read. Since the film thickness in the graph f is 5 nm, if the wavelength adjustment layer 2 is formed to have a film thickness of 5 nm, the preferable peak wavelength of the optical device 10 is shifted to the measurement wavelength 635 nm, and the antigen 12 can be preferentially detected.

As described above, the characteristic graph of the prototype optical device 30 is obtained and the preferable peak wavelength is identified. Next, the film thickness of the wavelength adjustment layer 2 is set based on the identified preferable peak wavelength and the measurement wavelength of the light source 22 and with reference to the second relationship (third step). Here, it is assumed that the wavelength adjustment layer 2 is made of SiON and has a refractive index of 3.5. When the preferable peak wavelength in the characteristic graph of the prototype optical device 30 is 630 nm and the measurement wavelength is 635 nm, it can be understood from the graph f that the film thickness of the wavelength adjustment layer 2 is preferably 5 nm to achieve the preferable peak wavelength matching the measurement wavelength of 635 nm. Thus, the preferable peak wavelength of the optical device 10 can be shifted to the measurement wavelength, and the presence of the antigen 12 can be detected with high sensitivity using the optical device 10 at the measurement wavelength. While the film thickness is set to be increased by 1 nm at a time in an example of the characteristic graph in FIG. 7, the thickness may be obtained by interpolation based on the specified peak wavelength.

Based on the set film thickness, the wavelength adjustment layer 2 is formed on the substrate 1, the reflective layer 3 is formed on the wavelength adjustment layer 2, and the optical waveguide layer 4 is formed on the reflective layer 3. The wavelength adjustment layer 2, the reflective layer 3, and the optical waveguide layer 4 may be formed by different film formation steps or may be formed by continuous film formation.

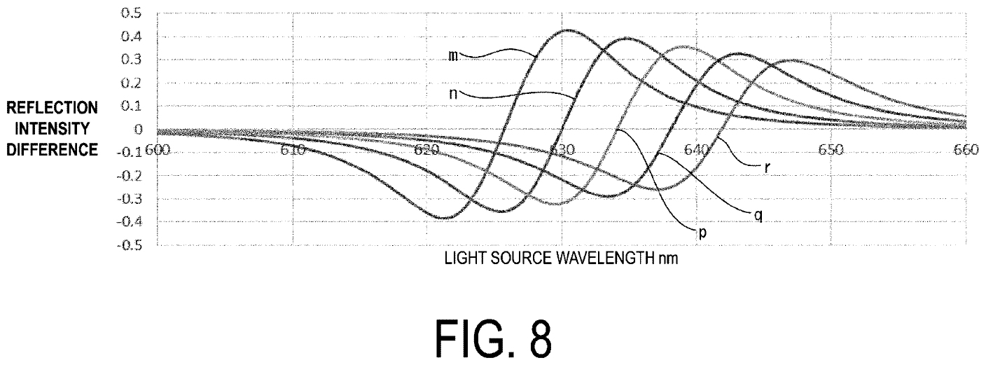

FIG. 8 illustrates examples of a plurality of characteristic graphs for different film thicknesses of the optical waveguide layer 4. The optical waveguide layer 4 in this example is made of SiO₂ and has a refractive index of 1.457. The incident angle is 67.5°. In FIG. 8, m, n, p, q, and r respectively indicate cases where the film thicknesses of the optical waveguide layer 4 are 350 nm, 355 nm, 360 nm, 365 nm, and 370 nm. As illustrated in FIG. 8, among the graphs of the respective film thicknesses, there is a difference in peak wavelength, and there is also a difference in the peak height (reflection intensity difference) unlike in the graphs in FIG. 7. When a characteristic graph is created for the manufactured prototype optical device 30 and the film thickness of the optical waveguide layer 4 is set so that the peak wavelength shifts to the measurement wavelength, the reflection intensity difference changes together with the peak wavelength. For example, when the peak wavelength is 630 nm as in the graph m, the film thickness is 350 nm and a large reflection intensity difference exceeding 0.4 is achieved. On the other hand, for example, when the peak wavelength is 635 nm as shown in the graph n, since the film thickness in the graph n is 355 nm, if the optical waveguide layer 4 is formed to have the film thickness of 355 nm, the peak wavelength 635 nm can be shifted to the measurement wavelength 630 nm. Still, the peak height falls below 0.4 to be lower than that in the case of the graph m. Therefore, it is understood that the presence of the antigen 12 cannot be detected with high sensitivity unlike in the case where the film thickness of the wavelength adjustment layer 2 is changed.

Although the detailed mechanism is not known, it is understood from the results illustrated in FIGS. 7 and 8 that the preferable peak wavelength can be shifted to a desired measurement wavelength without changing the peak height (reflection intensity difference) in the optical device 10 by adjusting the film thickness of the wavelength adjustment layer 2 instead of the optical waveguide layer 4.

As described above, according to the optical device 10 of the first embodiment, the preferable peak wavelength can be easily adjusted. That is, for the antibody 11 having specific optical characteristics, the optical characteristic can be easily adjusted through easy adjustment of the layer configuration of the optical device 10 with respect to variations in the characteristics due to manufacturing variations, to be suitable for detection of a change due to the capturing of the antigen 12. The optical device 10 can be manufactured with the film design of the prototype optical device 30 easily corrected through simple interposing of the wavelength adjustment layer 2, having a set film thickness, between the substrate 1 and the reflective layer 3 as a manufacturing condition of the optical device 10. Even after the film is designed to achieve desired characteristics, the optical device 10 can be easily corrected for variations in the characteristic that may occur, whereby the optical device 10 can be stably manufactured. The wavelength adjustment layer 2 whose film thickness is set can be formed under stable and easily manageable manufacturing conditions, and the detection performance of the prototype optical device 30 can be quickly reflected in the optical device 10.

In the first embodiment, the case where the antibody 11 is immobilized as the capturing body on the surface of the optical device 10 is described, but this should not be construed in a limiting sense. A nucleic acid or the like may be immobilized as a capturing body. The antigen 12 (specimen) recognized by the antibody 11 (capturing body) is not particularly limited, and may be any kind of protein. Although the case where the surface of the optical waveguide layer 4 has a silanol group as a specific functional group is described, this should not be construed in a limiting sense. For example, another specific functional group such as the above-mentioned carboxyl group may be formed on the surface of the optical waveguide layer 4.

In the first embodiment, when the characteristic graph is obtained, the comparison object in which the antigen 12 is not captured by the antibody 11 is water, but this should not be construed in a limiting sense. The comparison object may be any object whose reflection intensity changes before and after the antigen 12 (specimen) is captured by the antibody 11 (capturing body), and the liquid containing the specimen may be another liquid (solvent). In this case, various materials can be used as long the resonance point can be shifted without changing the intensity of each of the first reflection intensity and the second reflection intensity, that is, the preferable peak wavelength can be shifted without changing the reflection intensity difference in the characteristic graph.

In the first embodiment, as illustrated in FIG. 7, a case where characteristic graphs corresponding to a plurality of film thicknesses are held is described, but this should not be construed in a limiting sense. The second relationship corresponding to a plurality of film thicknesses may be held as a data table. Based on characteristic graphs corresponding to a plurality of film thicknesses, the film thickness may be expressed as a function of the peak wavelength with the light source wavelength being a constant.

In the first embodiment, the case where the wavelength adjustment layer 2 is provided between the substrate 1 and the reflective layer 3 is described, but this should not be construed in a limiting sense. The wavelength adjustment layer 2 may be provided between the reflective layer 3 and the optical waveguide layer 4. In this case, when the wavelength adjustment layer 2 is provided between the reflective layer 3 and the optical waveguide layer 4, the second relationship is obtained by changing the film thickness of the wavelength adjustment layer 2. As in a second embodiment described below, the wavelength adjustment layer 2 may be provided between the substrate 1 and the reflective layer 3 and between the reflective layer 3 and the optical waveguide layer 4. In this case, the second relationship may be obtained for a combination of different film thicknesses of both of the wavelength adjustment layers 2.

### Second Embodiment

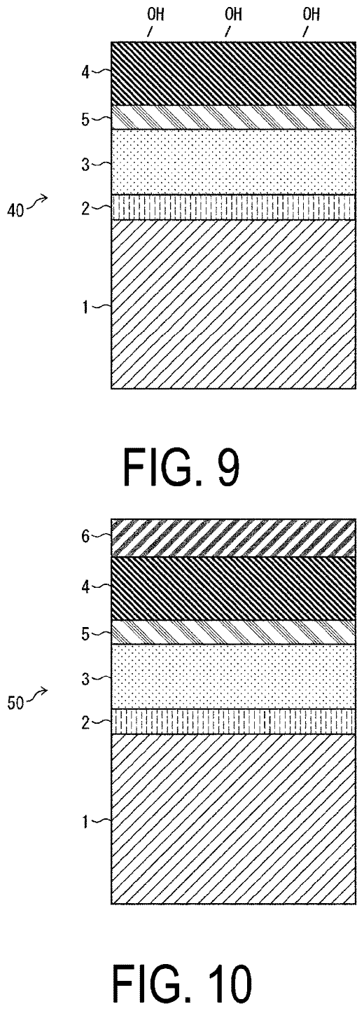

FIG. 9 is a cross-sectional view of an optical device 40 according to a second embodiment of the present disclosure. The optical device 40 includes the substrate 1, the wavelength adjustment layer 2, the reflective layer 3, a wavelength adjustment layer 5, and the optical waveguide layer 4. In the optical device 40, the wavelength adjustment layer 2, the reflective layer 3, the wavelength adjustment layer 5, and the optical waveguide layer 4 are formed in this order on the substrate 1.

The substrate 1, the reflective layer 3, and the optical waveguide layer 4 have the same configurations as those of the substrate 1, the reflective layer 3, and the optical waveguide layer 4 of the optical device 10 according to the first embodiment.

Wavelength Adjustment Layer The wavelength adjustment layer 2 has the same configuration as that of the wavelength adjustment layer 2 of the optical device of the first embodiment. The wavelength adjustment layer 2 is located on the substrate 1. Examples of the material of the wavelength adjustment layer 2 include SiON, SiN, and the like. The wavelength adjustment layer 2 may have a refractive index not less than 3 and not more than 3.7 when the light source wavelength is 632.8 nm, for example.

The wavelength adjustment layer 5 is located on the surface of the reflective layer 3 opposite to the surface on which the wavelength adjustment layer 2 is located. Examples of the material of the wavelength adjustment layer 5 include SiON, SiN, and the like. The material of the wavelength adjustment layer 5 may be the same as or different from the wavelength adjustment layer 2. The wavelength adjustment layer 5 may have a refractive index of not less than 3 and not more than 3.7 when the light source wavelength is 632.8 nm, for example.

Specifically, the wavelength adjustment layer 2 and the wavelength adjustment layer 5 shift the peak wavelength in a waveform indicating a first relationship between (i) the wavelength of light incident on the substrate 1 and (ii) the intensity of the light reflected by the surface of the reflective layer 3 on the optical waveguide layer 4 side or the surface of the optical waveguide layer 4 opposite to the reflective layer 3 under the total reflection condition.

Method of Setting Film Thickness of Wavelength Adjustment Layer For each of the optical devices 40 with different film thicknesses of the wavelength adjustment layer 2 and the wavelength adjustment layer 5, the second relationship between the preferable peak wavelength and film thicknesses of the wavelength adjustment layer 2 and the wavelength adjustment layer 5 in the characteristic graph is obtained in advance as a data table. Table 1 illustrates an example of the data table of the second relationship. Table 1 illustrates only some examples of combinations of the wavelength adjustment layer 2 and the wavelength adjustment layer 5. The data of a column for the case where the film thickness of the wavelength adjustment layer 5 is 0 nm indicates an example of the second relationship between the preferable peak wavelength and the thickness of the wavelength adjustment layer 2 in a case where only the wavelength adjustment layer 2 is formed. Similarly, the data of a row for the case where the film thickness of the wavelength adjustment layer 2 is 0 nm indicates an example of the second relationship between the preferable peak wavelength and the thickness of the wavelength adjustment layer 5 in a case where only the wavelength adjustment layer 5 is formed.

TABLE 1
Peak wavelength (nm)

| | | Wavelength adjustment layer 5 (nm) | | | | | | | | | | |
|---|---|---|---|---|---|---|---|---|---|---|---|---|
| | | 0 | 1 | 2 | 3 | 4 | 5 | 6 | 7 | 8 | 9 | 10 |
| Wavelength | 0 | 630 | 631 | 632 | 633 | 634 | 635 | 636 | 637 | 638 | 639 | 640 |
| adjustment | 1 | 631 | 632 | 633 | • | • | • | • | • | • | • | • |
| layer 2 | 2 | 632 | 633 | 634 | • | • | • | • | • | • | • | • |
| (nm) | 3 | 633 | • | • | • | • | • | • | • | • | • | • |
| | 4 | 634 | • | • | • | • | • | • | • | • | • | • |
| | 5 | 635 | • | • | • | • | • | • | • | • | • | • |
| | 6 | 636 | • | • | • | • | • | • | • | • | • | • |
| | 7 | 637 | • | • | • | • | • | • | • | • | • | • |
| | 8 | 638 | • | • | • | • | • | • | • | • | • | • |
| | 9 | 639 | • | • | • | • | • | • | • | • | • | • |
| | 10 | 640 | • | • | • | • | • | • | • | • | • | • |

The biosensor 20 is configured by using the prototype optical device 30, and a graph showing the second reflection intensity of water before the antigen 12 is captured by the antibody 11 and a graph showing the first reflection intensity when the antigen 12 is captured by the antibody 11 (graph of the first relationship) are obtained. A characteristic graph is obtained from these two graphs, and a preferable peak wavelength is obtained.

For example, when the preferable peak wavelength is 630 nm and the measurement wavelength is 634 nm, referring to Table 1, the peak wavelength of 630 nm can be shifted to the measurement wavelength of 634 nm by setting the film thickness of the wavelength adjustment layer 2 to 2 nm and the film thickness of the wavelength adjustment layer 5 to 2 nm.

According to the second embodiment, since the wavelength adjustment layer 2 and the wavelength adjustment layer 5 are provided on both sides of the reflective layer 3, it is possible to adjust the preferable peak wavelength more finely or in a larger range and improve the detection sensitivity of the detector 23 of the biosensor.

In the second embodiment, the case where the capturing body is the antibody 11 is described, but this should not be construed in a limiting sense. The comparison object for obtaining the characteristic graph is not limited to water.

In the second embodiment, the case where the second relationship between the peak wavelength and the film thicknesses of the wavelength adjustment layer 2 and the wavelength adjustment layer 5 is held as a data table is described. Alternatively, a characteristic graph corresponding to a plurality of film thicknesses of the wavelength adjustment layer 2 and the wavelength adjustment layer 5 may be held.

### Third Embodiment

FIG. 10 is a cross-sectional view of an optical device 50 according to a third embodiment of the present disclosure. The optical device 50 includes the substrate 1, the wavelength adjustment layer 2, the reflective layer 3, the wavelength adjustment layer 5, the optical waveguide layer 4, and a non-specific adsorption reduction layer 6. In the optical device 50, the wavelength adjustment layer 2, the reflective layer 3, the wavelength adjustment layer 5, the optical waveguide layer 4, and the non-specific adsorption reduction layer 6 are formed in this order on the substrate 1.

The substrate 1, the wavelength adjustment layer 2, the reflective layer 3, the optical waveguide layer 4, and the wavelength adjustment layer 5 have configurations similar to the configurations of the wavelength adjustment layer 2, the reflective layer 3, the optical waveguide layer 4, and the wavelength adjustment layer 5 of the optical device 40 according to the second embodiment. The film thicknesses of the wavelength adjustment layer 2 and the wavelength adjustment layer 5 are set by a setting method similar to the setting method for the film thicknesses of the wavelength adjustment layer 2 and the wavelength adjustment layer 5 of the optical device 40 of the second embodiment.

The non-specific adsorption reduction layer 6 is located on the optical waveguide layer 4, can be chemically modified, and can form a specific functional group for immobilizing a capturing body for specifically capturing a specimen on the surface. The non-specific adsorption reduction layer 6 has an optically translucent physical property and is formed so as to cover the entire optical waveguide layer 4. The non-specific adsorption reduction layer 6 is chemically and physically inactive, and adsorption of non-specific impurities to the surface of the non-specific adsorption reduction layer 6 is reduced. As a result, the optical device 50 has a surface that is inactive with respect to adsorption of impurities. The non-specific adsorption reduction layer 6 may contain C as the main material. Examples of the material of the non-specific adsorption reduction layer 6 include amorphous C (a-C) and/or SiC (a-SiC), and the like. When the optical waveguide layer 4 contains SiC, the non-specific adsorption reduction layer 6 is formed so as to be richer in C than the optical waveguide layer 4 and has a surface more inactive than the optical waveguide layer 4. As a result, the non-specific adsorption reduction layer 6 becomes a layer having an inactive surface as compared with the optical waveguide layer 4. The film thickness of the non-specific adsorption reduction layer 6 may be about 1 to 10 nm for the sake of reduction of the adsorption of non-specific impurities on the surface of the optical device 50 and reduction of an impact of near-field light on detection.

The non-specific adsorption reduction layer 6 is formed on the surface of the optical waveguide layer 4 by a film forming method such as bias Chemical Vapor Deposition (CVD), plasma CVD (p-CVD), or vapor deposition (Physical Vapor Deposition (PVD)) for example. The non-specific adsorption reduction layer 6 may be formed so as to cover the entire surface by, for example, growing the layer from an island shape to a layer shape by plasma CVD. The wavelength adjustment layer 2, the reflective layer 3, the optical waveguide layer 4, the wavelength adjustment layer 5, and the non-specific adsorption reduction layer 6 may be formed by separate film formation processes using a plurality of film formation apparatuses, or may be formed by continuous film formation using the same film formation apparatus.

On the surface of the non-specific adsorption reduction layer 6, a specific functional group for immobilizing a capturing body is formed by chemical modification. When the non-specific adsorption reduction layer 6 contains a-C and/or a-SiC, a carboxyl group is formed on the surface. When the surface of the non-specific adsorption reduction layer 6 is carboxylated, the surface activity can be controlled and the density of the carboxyl group can be adjusted by adjusting the concentration and time of the chemical treatment. That is, the carboxyl group can be formed on the surface of the non-specific adsorption reduction layer 6 in an amount required for immobilization of the capturing body. Since a silanol group can be formed as a specific functional group on the surface of the non-specific adsorption reduction layer 6, the silanol group may be appropriately and selectively used in consideration of the compatibility with the capturing body and the like.

The silanol group is exposed on the surface of the optical waveguide layer 4. According to the third embodiment of the present disclosure, since the optical waveguide layer 4 is covered with the non-specific adsorption reduction layer 6 to reduce unnecessary silanol groups exposed on the surface, non-specific adsorption of impurities other than the antibody 11 and the like to the silanol groups is reduced.

By forming the stable non-specific adsorption reduction layer 6 on the surface of the optical waveguide layer 4 containing an easily oxidizable silicon-based material, it is possible to reduce a change in the surface until the antibody 11 is immobilized on the surface of the optical device 50. As described above, by forming carboxyl groups (specific functional groups) in an amount required for immobilization of the antibody 11 on the surface of the non-specific adsorption reduction layer 6 and then immobilizing the antibody 11, it is possible to easily control the amount of immobilization of the antibody 11 and reduce non-specific adsorption of impurities. The surface of the non-specific adsorption reduction layer 6 after the immobilization of the antibody 11 is stable, and a change in the surface during storage of the optical device 50 is suppressed.

In the third embodiment, since the optical device 50 includes the wavelength adjustment layer 2 and the wavelength adjustment layer 5, even when a biosensor is configured using the optical device 50 and the light source wavelength is fixed, the optical device 50 can be easily corrected to desired characteristics for variations in the characteristics of the optical device 50 caused by manufacturing variations, and the antigen 12 can be detected with high sensitivity at the measurement wavelength. In addition, since the optical device 50 includes the non-specific adsorption reduction layer 6, non-specific adsorption of impurities is reduced, noise at the time of detection is reduced, and the antigen 12 can be satisfactorily detected.

In the third embodiment, the case where the optical device 50 includes the wavelength adjustment layer 2 and the wavelength adjustment layer 5 is described, but this should not be construed in a limiting sense. Any one of the wavelength adjustment layer 2 and the wavelength adjustment layer 5 may be provided.

The non-specific adsorption reduction layer 6 may not be formed on the entire surface of the optical waveguide layer 4, but may cover the surface of the optical waveguide layer 4 so as to expose a part thereof by controlling the degree of island-like growth of the film of the non-specific adsorption reduction layer 6. The silanol group on the surface of the underlying optical waveguide layer 4 in a portion not covered with the non-specific adsorption reduction layer 6 is directly exposed. Since the exposed silanol group can be used as a specific functional group for immobilization of the antibody 11, it is not necessary to form the carboxyl group by the carboxylation on the surface of the non-specific adsorption reduction layer 6.

### EXAMPLE

Hereinafter, examples of the present disclosure will be specifically described; however, the present disclosure is not limited to these examples.

### Example 1

As the substrate 1, a 1 mm glass substrate having a refractive index of 1.457 with respect to light having a wavelength of 625 nm was used. SiON was used to form the wavelength adjustment layer 2 having a refractive index of 3.5 with respect to light having a wavelength 625 nm and a film thickness of 5 nm on the substrate 1. On the wavelength adjustment layer 2, a-Si was used to form the reflective layer 3 having a refractive index of 3.889 with respect to light having a wavelength 625 nm and a film thickness of 200 nm. The optical waveguide layer 4 made of SiO₂ and having a refractive index of 1.457 with respect to light having a wavelength of 625 nm and a film thickness of 350 nm was formed on the reflective layer 3 to manufacture an optical device of Example 1.

Since the optical device of Example 1 has the wavelength adjustment layer 2, the presence of the antigen 12 can be detected with high sensitivity at the measurement wavelength.

### Example 2

As the substrate 1, a 1 mm glass substrate having a refractive index of 1.457 with respect to light having a wavelength of 625 nm was used. On the substrate 1, a-Si was used to form the reflective layer 3 having a refractive index of 3.889 with respect to light having a wavelength 625 nm and a film thickness of 200 nm. SiON was used to form the wavelength adjustment layer 5 having a refractive index of 3.5 with respect to light having a wavelength 625 nm and a film thickness of 5 nm on the reflective layer 3. On the wavelength adjustment layer 5, the optical waveguide layer 4 made of SiO₂ and having a refractive index of 1.457 with respect to light having a wavelength 625 nm and a film thickness of 350 nm was formed to manufacture an optical device of Example 2. This optical device corresponds to the optical device 10 having the configuration illustrated in FIG. 1 in which the wavelength adjustment layer 2 is formed between the reflective layer 3 and the optical waveguide layer 4.

Since the optical device of Example 2 has the wavelength adjustment layer 5, the presence of the antigen 12 can be detected with high sensitivity at the measurement wavelength.

### Example 3

As the substrate 1, a 1 mm glass substrate having a refractive index of 1.457 with respect to light having a wavelength of 625 nm was used. SiON was used to form the wavelength adjustment layer 2 having a refractive index of 3.5 with respect to light having a wavelength 625 nm and a film thickness of 5 nm on the substrate 1. On the wavelength adjustment layer 2, a-Si was used to form the reflective layer 3 having a refractive index of 3.889 with respect to light having a wavelength 625 nm and a film thickness of 200 nm. SiON was used to form the wavelength adjustment layer 5 having a refractive index of 3.5 with respect to light having a wavelength 625 nm and a film thickness of 5 nm on the reflective layer 3. On the wavelength adjustment layer 5, the optical waveguide layer 4 made of SiO₂ and having a refractive index of 1.457 with respect to light having a wavelength 625 nm and a film thickness of 350 nm was formed to manufacture the optical device 40 of Example 3.

Since the optical device 40 of Example 3 has the wavelength adjustment layer 2 and the wavelength adjustment layer 5, the presence of the antigen 12 can be detected with high sensitivity at the measurement wavelength.

In the present disclosure, the invention has been described above based on the various drawings and embodiments. However, the invention according to the present disclosure is not limited to each embodiment described above. That is, the embodiments of the invention according to the present disclosure can be modified in various ways within the scope illustrated in the present disclosure, and embodiments obtained by appropriately combining the technical means disclosed in different embodiments are also included in the technical scope of the invention according to the present disclosure. In other words, a person skilled in the art can easily make various variations or modifications based on the present disclosure. Note that these variations or modifications are included within the scope of the present disclosure.

1. An optical device comprising:
a substrate configured to transmit light;
a reflective layer disposed on the substrate;
an optical waveguide layer configured to propagate the transmitted light transmitted through the reflective layer or near-field light bled from the reflective layer, the optical waveguide layer being located on the reflective layer and having a surface provided with a functional group that immobilizes a capturing body that captures a specimen; and
a wavelength adjustment layer located on the substrate side, the optical waveguide layer side, or both the substrate side and the optical waveguide layer side of the reflective layer, and configured to shift a peak wavelength in a waveform indicating a first relationship between (i) a wavelength of the light and (ii) an intensity of the light reflected by a surface of the reflective layer on the optical waveguide layer side or a surface of the optical waveguide layer on a side opposite to the reflective layer under a total reflection condition.

2. The optical device according to claim 1, wherein the following relational expression is satisfied:
(refractive index of the reflective layer)>(refractive index of the wavelength adjustment layer)>(refractive index of the optical waveguide layer).

3. The optical device according to claim 1, wherein
a film thickness of the wavelength adjustment layer is smaller than a film thickness of the reflective layer.

4. The optical device according to claim 1, wherein
the wavelength adjustment layer contains SiON.

5. A biosensor comprising:
the optical device according to claim 1;
a light incidence mechanism configured to enable light to be incident the reflective layer, from a surface opposite to a surface of the substrate on which the reflective layer is located in the optical device; and
a light detection mechanism configured to detect the reflected light of the light reflected on a surface of the reflective layer on the side of the optical waveguide layer or a surface of the optical waveguide layer opposite to the reflective layer under the total reflection condition.

6. A method of setting a film thickness of the wavelength adjustment layer of the optical device according to claim 1, the method comprising:
measuring for an optical device not comprising the wavelength adjustment layer while changing the wavelength of the light, a first reflection intensity, obtained when the light is incident on the substrate with the specimen being captured by the capturing body, on a surface of the reflective layer on the side of the optical waveguide layer or a surface of the optical waveguide layer opposite to the reflective layer and a second reflection intensity, obtained when the light is incident on the substrate without the specimen being captured, on the surface of the reflective layer on the side of the optical waveguide layer or the surface of the optical waveguide layer opposite to the reflective layer;
identifying a peak wavelength in a characteristic waveform indicating a difference between the first reflection intensity and the second reflection intensity measured in the measuring of the first reflection intensity and the second reflection intensity; and
setting a film thickness of the wavelength adjustment layer by referring to a second relationship between the peak wavelength in the characteristic waveform and the film thickness obtained in advance, based on the peak wavelength identified in the identifying of the peak wavelength.
